(19) United States
(12) Patent Application Publication
(10) Pub. No.: US 2020/0268387 A1
(43) Pub. Date: Aug. 27, 2020

(54) PURSE STRING SUTURE DEVICE

(71) Applicant: Covidien LP, Mansfield, MA (US)

(72) Inventor: AVVLN Srinivasa Murthy Aravalli, Hyderabad (IN)

(21) Appl. No.: 16/780,511

(22) Filed: Feb. 3, 2020

Related U.S. Application Data
(60) Provisional application No. 62/810,975, filed on Feb. 27, 2019.

(51) Int. Cl. A61B 17/11

(52) U.S. Cl. CPC A61B 17/11; A61B 2017/0409; A61B 2017/1142

(57) ABSTRACT

A surgical applicator is useful in forming a purse string suture and may be used to, e.g., close internal anal structure or to narrow a passage for performing transanal dissection to create total mesorectal excision. The surgical applicator includes an actuation assembly and a reload operatively coupled with the actuation assembly.

### CROSS-REFERENCE TO RELATED APPLICATION

This application claims the benefit of and priority to U.S. Provisional Patent Application No. 62/810,975 filed Feb. 27, 2019, the entire disclosure of which is incorporated by reference herein.

### BACKGROUND

### Technical Field

The disclosure relates to a surgical device and, more particularly, to a surgical device for forming a purse string suture using surgical fasteners having an arcuate portion in conjunction with a suture.

### Background of Related Art

Purse string suture devices may include a pair of serrated tissue clamping jaws provided with teeth for clamping the tissue to be sutured therebetween. Such devices include needle passages which extend through the teeth on each jaw for receiving a needle attached to a suture to be threaded through the tissue. In use, the tissue to be sutured is clamped between the jaws and the needle is manually passed through the needle passages in both jaws to thread the suture through the tissue. Thereafter, the jaws are opened and the purse string suture is tightened and wrapped to draw the tissue together. With this type of device, a considerable amount of manual effort and dexterity is required to accomplish the purse string suturing technique. Also, in such devices, it is difficult to control the flow of tissue between the teeth because an insufficient amount of space is provided to gather the tissue clamped by the jaws.

### SUMMARY

In accordance with an embodiment of the disclosure, a surgical applicator for forming a purse string suture includes an actuation assembly and a reload operatively coupled with the actuation assembly. The reload includes an outer sleeve defining a lumen extending therethrough, an elongate core extending through the outer sleeve, an inner sleeve movably supported on the elongate core, a drive rod operatively coupled with the inner sleeve such that rotation of the drive rod causes axial displacement of the inner sleeve, a surgical fastener slidably supported on the elongate core, and a suture extending through the lumen of the outer sleeve and the surgical fastener. The inner sleeve is configured to engage the surgical fastener to eject the surgical fastener from the reload when the drive rod advances the inner sleeve.

In an embodiment, the surgical fastener may include an arcuate portion configured to be slidably supported on the elongate core.

In another embodiment, the surgical fastener may include an annular member configured to receive the suture therethrough.

In yet another embodiment, the elongate core may include a stopper configured to inhibit advancement of the surgical fastener.

In still yet another embodiment, the inner sleeve may be concentrically supported on the elongate core.

In a further embodiment, the lumen of the outer sleeve may have a profile configured to receive the surgical fastener.

In an embodiment, the lumen may have an oblong transverse cross-sectional profile.

In another embodiment, the surgical applicator may further include an elongate member detachably supporting the reload thereon. The elongate member may include an actuation shaft operatively coupled to the actuation assembly and the drive rod of the reload.

In an embodiment, the drive rod may have a proximal portion defining a cavity having a cross-section complementary to a cross-section of the actuation shaft of the elongate member.

In accordance with another embodiment of the disclosure, a reload for use with a surgical applicator for forming a purse string suture includes an outer sleeve defining a lumen extending therethrough, an elongate core extending through the outer sleeve, an inner sleeve movably supported on the elongate core, a drive rod operatively coupled with the inner sleeve such that rotation of the drive rod causes axial displacement of the inner sleeve, a plurality of surgical fasteners mounted on the elongate core, and a suture extending through the lumen of the outer sleeve and the plurality of surgical fasteners. The inner sleeve is configured to engage a proximal-most surgical fastener such that rotation of the drive rod advances a distal-most surgical fastener out of the reload.

In an embodiment, at least a portion of the elongate core may be disposed distal of a distal end of the outer sleeve.

In another embodiment, the elongate core may include an outer surface having a stopper frictionally engaging a distal-most surgical fastener.

In yet another embodiment, the surgical fastener may include a body having an arcuate portion configured to be slidably supported on the elongate core, and a pair of arms expandable to engage tissue.

In accordance with yet another embodiment of the disclosure, a surgical applicator for forming a purse string suture includes a handle assembly and a reload. The handle assembly includes a motor including an output shaft and an actuator operatively coupled with the motor. The reload is operatively coupled with the actuation assembly. The reload includes an outer sleeve defining a lumen extending therethrough, an elongate core extending through the lumen of the outer sleeve, an inner sleeve movably supported on the elongate core, a drive rod operatively coupled with the inner sleeve such that rotation of the drive rod causes axial displacement of the inner sleeve, first and second surgical fasteners slidably supported on the elongate core, and a suture extending through the lumen of the outer sleeve and the first and second surgical fasteners. The inner sleeve is configured to engage the first surgical fastener such that rotation of the drive rod advances the first and second surgical fasteners.

In an embodiment, the inner sleeve may be threadably coupled to the drive rod.

In another embodiment, the first and second surgical fasteners may be disposed within the outer sleeve such that when the actuator of the handle assembly actuates the motor, the inner sleeve engages the first surgical fastener, which, in turn, advances the second surgical fastener out of the reload.

In yet another embodiment, the handle assembly may further include a battery.

### BRIEF DESCRIPTION OF THE DRAWINGS

Various embodiments of the disclosure are described hereinbelow with reference to the drawings, wherein:

FIG. 1 is a perspective view of a surgical applicator in accordance with an embodiment of the disclosure;

FIG. 1A is a partial side cross-sectional view of an elongate member of the surgical applicator of FIG. 1;

FIG. 2 is a schematic view of a reload of the surgical applicator of FIG. 1;

FIG. 3 is a perspective view of a suture and a plurality of surgical fasteners of FIG. 2;

FIG. 4 are side views of various embodiments of surgical fasteners for use with the surgical applicator of FIG. 1;

FIGS. 5 and 6 are top views of another surgical fastener for use with the surgical applicator of FIG. 1, illustrating use thereof;

FIGS. 7 and 8 are top views of yet another embodiment of a surgical fastener for use with the surgical applicator of FIG. 1, illustrating use thereof; and

FIG. 9 is a top view of the suture and the plurality of surgical fasteners of FIG. 3, illustrating use in a surgical site.

### DETAILED DESCRIPTION

Embodiments of the disclosure will now be described in detail with reference to the drawings, in which like reference numerals designate identical or corresponding elements in each of the several views. As used herein, the term “distal,” as is conventional, will refer to that portion of the instrument, apparatus, device or component thereof which is farther from the user while, the term “proximal,” will refer to that portion of the instrument, apparatus, device or component thereof which is closer to the user. In the following description, well-known functions or constructions are not described in detail to avoid obscuring the disclosure in unnecessary detail.

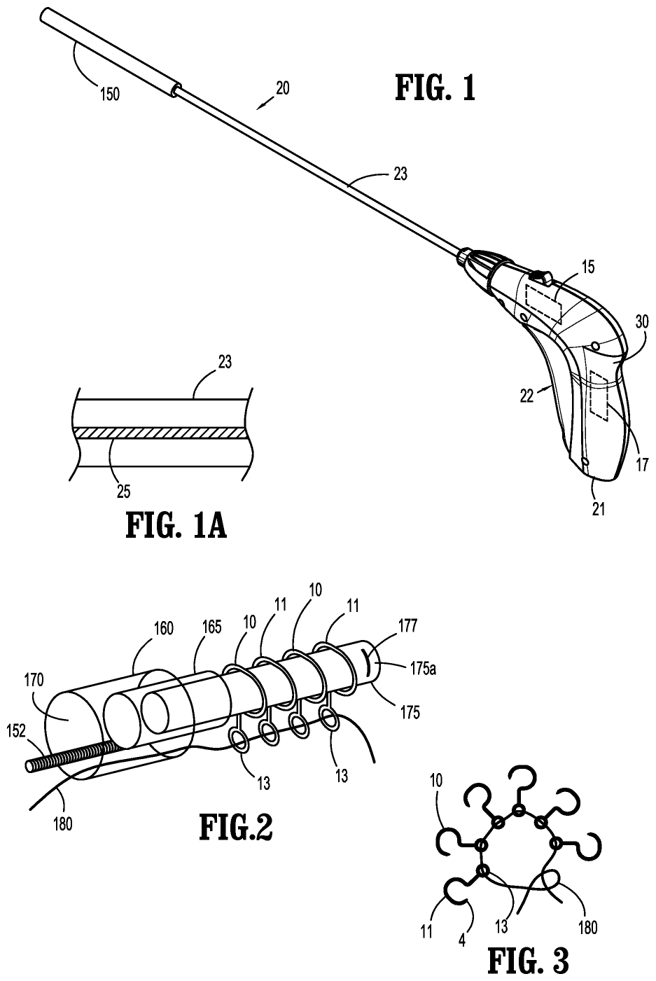

As illustrated in FIG. 1, there is illustrated a surgical applicator 20 configured to apply surgical fasteners 10 (FIG. 2) in conjunction with a suture 180 (FIG. 2) to form a purse string suture. For example, the surgical applicator 20 may be utilized in transanal total mesorectal excision (TaTME) for removal of low rectal and ultra-low rectal tumors and preservation of anal sphincters to avoid permanent stomas. In particular, the surgical applicator 20 may be utilized in forming surgical stitches used to close internal anal structure or to narrow a passage for performing further transanal dissection to create total mesorectal excision. The surgical applicator 20 generally includes a handle assembly 30 including a pistol grip handle 21 and an actuator 22, an elongate member 23 extending distally from the handle assembly 30, and a reload 150 releasably coupled to the elongate member 23. An outer diameter of the reload 150 and the elongate member 23 may be dimensioned for use with standard trocars or laparoscopic devices for minimally invasive entry into an opening in tissue. The reload 150 contains a plurality of serially arranged surgical fasteners 10. Actuation of the actuator 22 ejects a surgical fastener 10 from the reload 150.

As illustrated in FIGS. 1 and 1A, the handle assembly 30 includes a motor 15 (shown in phantom) configured to provide rotational output to a drive rod 152 (FIG. 2) of the reload 150. The handle assembly 30 may further include, e.g., an internal battery 17 (shown in phantom), to supply power to the motor 15. The actuator 22 is operatively coupled to the motor 15 to actuate the motor 15. The elongate member 23 includes an actuation shaft 25 extending through the elongate member 23. In particular, an output shaft of the motor 15 is rotatably coupled with the actuation shaft 25 for concomitant rotation therewith. The actuation shaft 25 is operatively coupled with the drive rod 152 of the reload 150 for concomitant rotation therewith. For example, the drive rod 152 includes a proximal section (not shown) configured to operatively engage the actuation shaft 25 of the elongate member 23 such that actuation of the motor 15 (FIG. 1) of the handle assembly 30 imparts rotation to the drive rod 152. For example, the proximal section of the drive rod 152 may include a cavity having a cross-section complementary to a cross-section of the actuation shaft 25 such that rotation of actuation shaft 25 causes concomitant rotation of the drive rod 152. In this manner, actuation of the motor 15 of the handle assembly 130 imparts concomitant rotation to the drive rod 152.

As illustrated in FIGS. 1A and 2, the reload 150 is releasably attached to the elongate member 23. The reload 150 includes an outer sleeve 160 defining a lumen 170 therethrough, an elongate core 175 extending through the lumen 170, an inner sleeve 165 slidably mounted on the elongate core 175, and the drive rod 152 rotatably supported within the lumen 170. The inner sleeve 165 may be, e.g., concentrically supported on the elongate core 175. In particular, the drive rod 152 is operatively coupled with the inner sleeve 165 such that rotation of the drive rod 152 causes axial displacement of the inner sleeve 165. For example, the drive rod 152 and the inner sleeve 165 may be threadably coupled.

As illustrated in FIGS. 2 and 3, a plurality of surgical fasteners 10 is mounted on the elongate core 175 in sequence. In particular, the elongate core 175 may have a sufficient length extending out of the outer sleeve 160 to support multiple surgical fasteners 10 external to the outer sleeve 160. Each surgical fastener 10 includes an annular member 13 dimensioned to receive a suture 180 therethrough and an arcuate portion 11 configured to be slidably mounted on the elongate core 175 independent of the annular member 13. The suture 180 extends through the lumen 170 of the outer sleeve 160 of the reload 150 and through the annular members 13 of the surgical fasteners 10. The arcuate portion 11 terminates in a sharp tissue penetrating point 4.

The entire elongate core 175 may be disposed within the outer sleeve 160. However, as discussed hereinabove, at least a portion of the elongate core 175 may extend distally from the outer sleeve 160. Under such a configuration, a distal-most surgical fastener 10 may be disposed external to the outer sleeve 160 to facilitate ejection of the surgical fastener 10 from the reload 150. However, it is contemplated that a plurality of surgical fasteners 10 may be external to the outer sleeve 160. The elongate core 175 may include a stopper 177 protruding from an outer surface 175a of the elongate core 175. The stopper 177 is configured to, e.g., frictionally retain the surgical fastener 10 on the elongate core 175, e.g., inhibit the surgical fastener 10 from being ejected from the reload 150. However, actuation of the inner sleeve 165, which advances the distal-most surgical fastener 10, overcomes, e.g., the frictional force to retain surgical fastener 10 on the elongate core 175, thereby releasing the surgical fastener 10 from the elongate core 175. It is contemplated that the number of stoppers 177 and the spacing between the stoppers 177 may be tailored to the surgical procedure being performed.

The inner sleeve 165 is slidably supported about the elongate core 175. In particular, the inner sleeve 165 is configured to engage at least the arcuate portion 11 of a surgical fastener 10 disposed on the elongate core 175 such that when the inner sleeve 165 is advanced, the inner sleeve 165 advances a surgical fastener 10 out of the reload 150. Each actuation may be calibrated to eject a single surgical fastener 10 from the reload 150. For example, the distal-most surgical fastener 10 may be placed between two stoppers 177.

The passage 170 of the outer sleeve 160 has a cross-section configured to receive the surgical fasteners 10 and the inner sleeve 165. For example, the outer sleeve 160 may have an oblong shape, e.g., an oblong transverse cross-sectional profile. The inner sleeve 165 may have an annular configuration that is, e.g., substantially identical to the configuration of the elongate core 175.

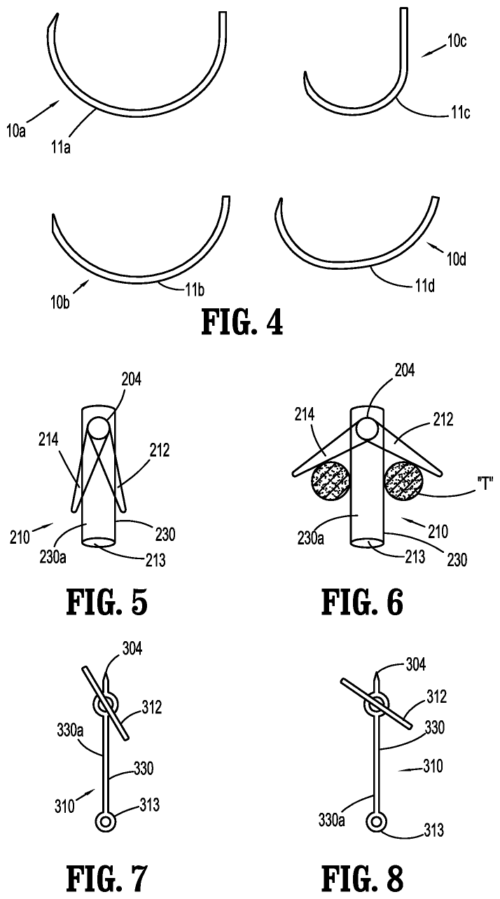

As illustrated in FIG. 4, the arcuate portion 11 of the surgical fastener 10 may include various arcuate profiles that may be supported on the elongate core 175 (FIG. 2). For example, a surgical fastener 10a in accordance with another embodiment of the disclosure, may include an arcuate portion 11a defining a ⅝ of a circle. In another embodiment, a surgical fastener 10b may include an arcuate portion 11b defining a half circle. In another embodiment, a surgical fastener 10c may include an arcuate portion 11c having a J-shape. Alternatively, a surgical fastener 10d in accordance with another embodiment of the disclosure may include an arcuate portion 11d having a compound curve profile.

As illustrated in FIGS. 5 and 6, a surgical fastener 210 in accordance with another embodiment of the disclosure for use with the surgical applicator 20 may include a body 230 having a sharp tissue penetrating point 204 and a pair of arms 212, 214 projecting in a reverse direction to the sharp tissue penetrating point 204. In addition, the body 230 defines a bore 213 dimensioned to receive the suture 180 (FIG. 2) therethrough. The pair of arms 212, 214 is transitionable between a retracted position (FIG. 5) for facilitating insertion through tissue and an expanded position (FIG. 6) for enhancing anchoring of the surgical fastener 210 in tissue. In this manner, the transitionable arms 212, 214 enhance anchoring of the surgical fastener 210 in tissue while reducing trauma to tissue during insertion thereof.

As illustrated in FIGS. 7 and 8, a surgical fastener 310 in accordance with another embodiment of the disclosure includes a body 330 having a sharp tissue penetrating point 304 and an arm 312 pivotally coupled to the body 330. In addition, the body 330 includes an annular member 313 dimensioned to receive the suture 180 therethrough. The arm 312 is transitionable between a retracted position to facilitate insertion through tissue (FIG. 7) and an expanded position (FIG. 8), in which, the arm 312 projects in a reverse direction to the sharp tissue penetrating point 304. In this manner, the arm 312 enhances anchoring of the surgical fastener 310 in tissue, while reducing trauma to tissue during insertion thereof.

The surgical fasteners 10, 210, 310 may be made from, e.g., semi-stiff implantable wire, such as titanium. Alternatively, the surgical fasteners 10, 210, 310 may include plastic or absorbable materials. Examples of materials that can be used in constructing the body may include titanium, titanium alloys, stainless steel, nickel, chrome alloys and any other biocompatible implantable metals. Alternatively, other options for materials are liquid crystal polymers, HDPE, polyglycolic acid, and polyglycolid hydroxgacetic acid. At least a portion of the surgical fasteners 10, 210, 310 may be coated with a biocompatible lubricious material that provides for easier delivery of the surgical fastener 10, 210, 310 into tissue.

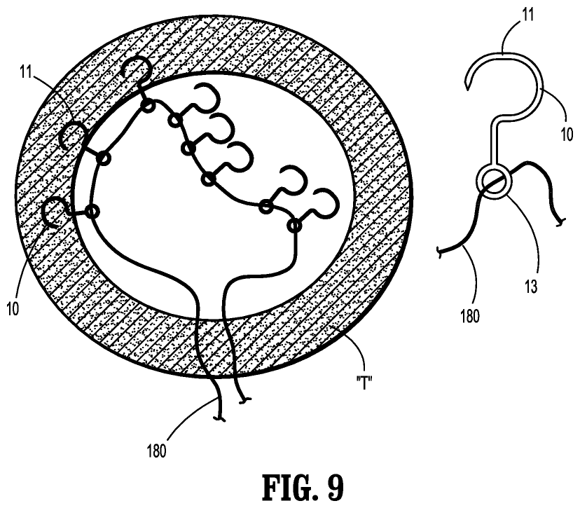

As illustrated in FIG. 9, in use, the surgical applicator 20 containing the surgical fasteners 10 is positioned adjacent the opening “O” in tissue “T” such that the surgical fasteners 10 may be applied to peripheral portions of the opening “O” in tissue “T.” Thereafter, the actuator 22 is actuated to cause rotation of the drive rod 152 relative to the inner sleeve 165 which, in turn, drives the surgical fastener 10 out of the reload 150. At this time, the clinician may use grasper (not shown) to hook the arcuate portion 11 of the surgical fastener 10 into tissue “T”. The clinician repeats the process until there are sufficient number of the surgical fasteners 10 surrounding the opening “O” of tissue “T” to provide stable anchoring when opposite ends of the suture 180 (FIG. 3) are pulled to close the opening “O.” Once the desired number of surgical fasteners 10 are anchored on a peripheral portion of the opening “O,” the opposing ends of the suture 180 are pulled by the clinician to close the opening “O.” In this manner, the formation of a purse string suture is simplified by, e.g., eliminating the need for complicated maneuvering of a needle inside an anal canal, which may reduce injuries to tissue. While the suture 180 may be inserted through the passage 170 of the outer sleeve 160 of the reload 150 and through the annular members 13 of the surgical fasteners 10 prior to the surgical procedure, it is also contemplated that the clinician may place the suture 180 through the annular member 13 after the surgical fastener 10 has been hooked onto tissue “T”.

Although the illustrative embodiments of the disclosure have been described herein with reference to the accompanying drawings, the above description, disclosure, and figures should not be construed as limiting, but merely as exemplifications of particular embodiments. For example, while a powered actuation utilizing a motor has been described in this disclosure, it is also envisioned that a mechanically triggered actuation assembly may be utilized to provide rotational output to the drive rod 152 of the reload 150. In addition, it is further contemplated that the surgical applicator 20 may be adapted for use in robotic surgery.

It is also to be appreciated that the disclosure may be utilized in a number of applications including ligating tissue, hernia mesh repair, and in conjunction with implant drug delivery systems or procedures involving positioning of surgical or implantable devices in patients. It is to be understood, therefore, that the disclosure is not limited to those precise embodiments, and that various other changes and modifications may be effected therein by one skilled in the art without departing from the scope or spirit of the disclosure.

What is claimed is:

1. A surgical applicator for forming a purse string suture, the surgical applicator comprising:
an actuation assembly; and
a reload operatively coupled with the actuation assembly, the reload including:
an outer sleeve defining a lumen extending therethrough;
an elongate core extending through the outer sleeve;
an inner sleeve movably supported on the elongate core;
a drive rod operatively coupled with the inner sleeve such that rotation of the drive rod causes axial displacement of the inner sleeve;
a surgical fastener slidably supported on the elongate core, the inner sleeve configured to engage the surgical fastener to eject the surgical fastener from the reload when the drive rod advances the inner sleeve; and
a suture extending through the lumen of the outer sleeve and the surgical fastener.

2. The surgical applicator according to claim 1, wherein the surgical fastener includes an arcuate portion configured to be slidably supported on the elongate core.

3. The surgical applicator according to claim 2, wherein the surgical fastener includes an annular member configured to receive the suture therethrough.

4. The surgical applicator according to claim 2, wherein the elongate core includes a stopper configured to inhibit advancement of the surgical fastener.

5. The surgical applicator according to claim 1, wherein the inner sleeve is concentrically supported on the elongate core.

6. The surgical applicator according to claim 1, wherein the lumen of the outer sleeve has a profile configured to receive the surgical fastener.

7. The surgical applicator according to claim 6, wherein the lumen has as oblong transverse cross-sectional profile.

8. The surgical applicator according to claim 1, further including an elongate member detachably supporting the reload thereon, the elongate member including an actuation shaft operatively coupled to the actuation assembly and the drive rod of the reload.

9. The surgical applicator according to claim 8, wherein the drive rod has a proximal portion defining a cavity having a cross-section complementary to a cross-section of the actuation shaft of the elongate member.

10. A reload for use with a surgical applicator for forming a purse string suture, the reload comprising:
an outer sleeve defining a lumen extending therethrough;
an elongate core extending through the outer sleeve;
an inner sleeve movably supported on the elongate core;
a drive rod operatively coupled with the inner sleeve such that rotation of the drive rod causes axial displacement of the inner sleeve;
a plurality of surgical fasteners mounted on the elongate core, the inner sleeve configured to engage a proximal-most surgical fastener such that rotation of the drive rod advances a distal-most surgical fastener out of the reload; and
a suture extending through the lumen of the outer sleeve and the plurality of surgical fasteners.

11. The reload according to claim 10, wherein the plurality of surgical fasteners is disposed distal of the inner sleeve.

12. The reload according to claim 11, wherein at least a portion of the elongate core is disposed distal of a distal end of the outer sleeve.

13. The reload according to claim 10, wherein the elongate core includes an outer surface having a stopper frictionally engaging a distal-most surgical fastener.

14. The reload according to claim 10, wherein each surgical fastener includes an annular member configured to receive the suture therethrough and an arcuate portion configured to be slidably mounted on the elongate core independent of the annular member.

15. The reload according to claim 10, wherein the surgical fastener includes a body having an arcuate portion configured to be slidably supported on the elongate core, and a pair of arms expandable to engage tissue.

16. A surgical applicator for forming a purse string suture, the surgical applicator comprising:
a handle assembly including:
a motor including an output shaft; and
an actuator operatively coupled with the motor; and
a reload operatively coupled with the actuation assembly, the reload including:
an outer sleeve defining a lumen extending therethrough;
an elongate core extending through the lumen of the outer sleeve;
an inner sleeve movably supported on the elongate core;
a drive rod operatively coupled with the inner sleeve such that rotation of the drive rod causes axial displacement of the inner sleeve;
first and second surgical fasteners slidably supported on the elongate core, the inner sleeve configured to engage the first surgical fastener such that rotation of the drive rod advances the first and second surgical fasteners; and
a suture extending through the lumen of the outer sleeve and the first and second surgical fasteners.

17. The surgical applicator according to claim 16, wherein the inner sleeve is threadably coupled to the drive rod.

18. The surgical applicator according to claim 16, wherein the first and second surgical fasteners are disposed within the outer sleeve such that when the actuator of the handle assembly actuates the motor, the inner sleeve engages the first surgical fastener, which, in turn, advances the second surgical fastener out of the reload.

19. The surgical applicator according to claim 18, wherein the suture extends through the lumen of the outer sleeve and the annular members of the plurality of surgical fasteners.

20. The surgical applicator according to claim 16, wherein the handle assembly further includes a battery.
